(19) United States
(12) Patent Application Publication
(10) Pub. No.: US 2005/0031487 A1
(43) Pub. Date: Feb. 10, 2005

(54) Use of high-purity chlorine dioxide gas to inactivate finely milled, humidification-resistant "weaponized" spores

(76) Inventors: Aaron A. Rosenblatt, New York, NY (US); Thomas E. NcWorter, Allentown, PA (US)

Correspondence Address: RATNERPRESTIA, P O BOX 980, VALLEY FORGE, PA, 19482-0980, US

(21) Appl. No.: 10/498,666

(22) PCT Filed: Dec. 17, 2002
(86) PCT No.: PCT/US02/40189

Related U.S. Application Data
(60) Provisional application No. 60/341,629, filed on Dec. 17, 2001.

(51) Int. Cl. A61L 2/20

(52) U.S. Cl. 422/37

(57) ABSTRACT

Weaponized spores are inactivated by subjecting the spores to humidification to both satisfy water uptake of any fillers present with the spores and humidify the spores, followed by sterilization with chlorine dioxide.

### BACKGROUND OF THE INVENTION

The present invention pertains to decontamination of facilities and their contents, especially those contaminated with weapons grade bio-warfare agents.

Recent terrorist use of Anthrax spores as a bio-warfare agent (BWA) has contaminated a number of buildings, including postal facilities, mailrooms and a Senate office building as well as considerable quantities of mail. These facilities and furniture, fixtures, equipment and materials contained therein require suitable decontamination. Decontaminating methods include the use of foams and liquid anti-microbial agents, such as bleach, to disinfect surfaces. For decontamination of facilities or materials that may have been subject to pathogens which can aerosolize, (such as the finely-divided Anthrax spores employed in recent bio-terror incidents) it is advantageous to employ a decontaminating gas. Gas molecules can decontaminate any aerosolized, airborne pathogens, and also can diffuse thoroughly through all the cracks and crevices in a facility and reach any surface that might have been reached by the target pathogen(s). Whatever the decontamination method used, it is important that such method be applied under conditions where it can achieve the intended result.

It is well known in the medical device and pharmaceutical industries that, when undertaking sterilization/decontamination, it is essential to understand thoroughly the critical parameters within which the target pathogen will be destroyed. Typically, there is some tradeoff between critical parameters—time, relative humidity, temperature and gas concentration—but the relationships are not necessarily linear. It is important to establish the resistance of the target pathogen(s) to the decontaminating agent. It is customary to use a non-pathogenic surrogate organism to model the expected behavior of a highly pathogenic one. Bacillus subtilis is widely recognized as an appropriate surrogate for chemo-sterilization resistant organisms, such as B. anthracis (Anthrax). Optimally, the surrogates (a/k/a, “Biological Indicators” or “BI's”) have been correlated with the particular types and strains of pathogens being targeted.

When pathogens are intended for use as biological warfare agents (BWA), as in recent cases of mail-borne Anthrax, the pathogens may be specially-prepared (“weaponized”) so that they can aerosolize and be inhaled by victims. Weaponized spores, such as those that cause the particularly deadly “inhalation Anthrax”, have several distinguishing characteristics: (1) They are small—reportedly on the order of 1-3 microns in size. This facilitates their easy dispersion, and ready entry deep into victims' lungs. (2) The particles remain discreet—they don't “clump” together—and are able to be aerosolized; and (3) in at least some cases, there is a high concentration of spores per unit of material. The weaponized Anthrax prepared by the US Army Bio-warfare program reportedly contains about one trillion—i.e., 10¹²—spores per gram.

Weaponizing may involve several steps, including drying and milling spores to the desired size. However, several factors, including the natural hygroscopicity of spores and electrostatic surface charge that may be associated with milling fine particles, may cause the finely milled spores to clump together. In order to keep weaponized spores finely divided and to prevent “clumping”, they may be treated in various ways. For example, they may be mixed with a substantial portion of finely divided dry materials (fillers) that have a stronger affinity for moisture than do the dessicated spores. They also may be surface modified to remove the electrostatic charge on the surface of the material. Such processes would help prevent clumping and would facilitate aerosolization. However, these procedures would also make much more difficult the humidification of the dry, fine-milled spores. Since humidification is a critical parameter in chemo-sterilization, weaponization that renders spores resistant to humidification makes them less susceptible (i.e., resistant) to chemo-sterlization processes.

### BRIEF SUMMARY OF THE INVENTION

It has been discovered that in order to inactivate weaponized spores, the spores and any filler materials mixed with the spores must be thoroughly humidified before the spores are exposed to a suitable decontaminant such as chlorine dioxide.

Therefore, in one aspect the present invention is a method for inactivating weaponized spores comprising the steps of, humidifying the spore preparation (i.e. processed spores plus filler materials) under conditions of time, temperature and relative humidity of a humidifying atmosphere which both substantially satisfies the water-uptake of any fillers admixed with the spores and humidifies the spores and, subjecting the humidified spore preparation to a sterilizing concentration of chlorine dioxide gas.

### DETAILED DESCRIPTION OF THE INVENTION

Spores in any form are generally grown from a known bacterium in a fermentation process. Spores such as B. anthracis tend to clump together when in finely divided form and thus will not aerosolize readily. It is believed that the spores can be made able to aerosolize by mixing the spores with a dry filler (e.g., silica gel, kaolin or bentonite clay) and then milling the mixture to obtain a finely divided powder of spores and filler. The finely divided spores and filler mixture will readily aerosolize and not stick to surfaces, and will readily stay airborne when agitated.

The weaponized spores present a difficult problem for any decontaminate to achieve kill.

Recent attempts to decontaminate facilities contaminated with Anthrax spores (such as the Hart Senate office building in Washington, D.C.) have reportedly utilized the standard B. subtilis bio-indicators, a/k/a “spore strips”, that are routinely employed in the medical device and pharmaceutical industries. These spore strips contain approximately one million (i.e., 10⁶) non-pathogenic spores on a cellulose substrate. They are in a “natural” state, that is, they have not been treated so as to make them equivalent to weaponized spores.

If standard BI's are more susceptible to a sterilization/decontamination regime than the weaponized target organism for which they are serving as surrogates, it is likely that a false sense of security can result, with the assumption that a facility has been rid of pathogens, such as weaponized Anthrax, when the pathogen may indeed still be viable and able to cause deadly illness. One way to facilitate the necessary correlation between a surrogate BI and the target pathogen is to treat non-pathogenic spores with a weaponizing process similar to that used to produce the weaponized pathogen that is the “real” target, imparting comparable characteristics re their resistance to decontamination. These specially-processed non-pathogenic organisms can then serve as proper surrogates for the target pathogen, that is, these formulations are essentially “weaponized”. They are difficult to humidify, and are more resistant to sterilization regimes than their untreated counterparts that are used as standard bio-indicators.

The use of chlorine dioxide gas for the chemo-sterilization of medical devices is well known. (Rosenblatt et al. U.S. Pat. No. 4,681,739). For medical device sterilization, chlorine dioxide sterilization protocols were developed using commercial B. subtilis bio-indicators (spore strips) with a spore concentration of 10⁶, the industry standard.

Using non-pathogenic, weaponized spore preparations, of about 5.5×10¹⁰ spores per gram (2.75% by weight in kaolin filler; approximate particle size of 1-3 microns), experiments were conducted comparing the inactivation of standard commercial spore strips with the “weaponized” BI's.

In one test, standard BI's and weaponized BI's were placed in paper envelopes. The envelopes were placed in a glass reactor vessel and subjected to two cycles consisting of:
1. Drawing a vacuum of about 27″ Hg.
2. Introducing nitrogen at approximately 75% relative humidity
3. Holding the humidified nitrogen at about atmospheric pressure for one hour

The samples were then subjected to 3 cycles consisting of:
1. Drawing a vacuum of about 27″ Hg.
2. Filling the reactor with chlorine dioxide gas at about 10,000 ppm in nitrogen at about atmospheric pressure.
3. Holding the gas in the reactor for 1 hour.

At the end of the 3^{rd} exposure to chlorine dioxide, the chamber was purged by twice:
1. Drawing a vacuum of about 27″ Hg
2. Filling the reactor to about atmospheric pressure with nitrogen at about 75% relative humidity

After this process, tests were performed to determine viability of all BI's. Three of 4 of the standard BI's in envelopes were completely inactivated; while one showed some sign of viable spores, probably as a result of contamination during handling. The weaponized BI's, in envelopes, showed little, if any, reduction in viability. That is, the cycle that achieved at least 6-log inactivation of standard BI's was ineffective against weaponized spores. The envelopes were weighed before and after treatment; weight was substantially unaffected.

In a second experiment, the weaponized spores in envelopes were pre-humidified at approximately 100% relative humidity (RH), 35° C., for 18.5 hours. At a chlorine dioxide concentration of 10,000 ppm for an exposure time of 3 hours with relative humidity at estimated 100%, the weaponized BI's, placed in paper envelopes, were completely inactivated (at least 10-log kill). The envelopes were weighed before and after treatment; weight increased about 20%—attributable to water uptake.

Clearly, humidification of the weaponized preparation is necessary to achieve kill.

It is believed that the water-uptake capacity of the “filler” (kaolin, bentonite clay, silica gel) needs to be substantially satisfied before the dessicated spores will humidify sufficiently to be vulnerable to inactivation by chlorine dioxide gas.

1. A method for inactivating weaponized spores comprising the steps of:
humidifying said spores under conditions of time, temperature and relative humidity of a humidifying atmosphere which both substantially satisfies the water-uptake of any fillers admixed with said spores and humidifies said spores; and subjecting said humidified spores and fillers to a sterilizing concentration of chlorine dioxide gas.

2. A method according to claim 1 including the step of humidifying said atmospheres to a level of 100% relative humidify.

3. A method according to claim 1 including the step of carrying out said humidifying at a temperature at or above 35° C.

4. A method according to claim 1 including the step of carrying out said humidifying steps for at least 18 hours.

5. A method according to claim 1 including the step of humidifying said spores under an atmosphere having 100% relative humidity at a temperature at or above 35° C. for at least 18 hours.
